(19) United States
(12) Patent Application Publication
(10) Pub. No.: US 2015/0208964 A1
(43) Pub. Date: Jul. 30, 2015

(54) SYSTEMS AND METHODS FOR DETERMINING RESPIRATION INFORMATION

(71) Applicant: Covidien LP, Mansfield, MA (US)

(72) Inventors: Paul Stanley Addison, Edinburgh (GB); James Nicholas Watson, Dunfermline (GB)

(21) Appl. No.: 14/599,896

(22) Filed: Jan. 19, 2015

Related U.S. Application Data
(60) Provisional application No. 61/932,167, filed on Jan. 27, 2014.

(51) Int. Cl. A61B 5/1455; A61B 5/00; A61B 5/0205

(52) U.S. Cl. CPC A61B 5/14551; A61B 5/0205; A61B 5/7221; A61B 5/7278; A61B 5/02416

(57) ABSTRACT

Systems and methods are provided for determining respiration information. Respiration information is determined from physiological signals responsive to regional oxygen saturation information. Respiration information is determined based on any of the amplitude, frequency, or baseline components of the physiological signals.

### CROSS-REFERENCE TO RELATED APPLICATION

The present disclosure claims priority to U.S. Provisional Application No. 61/932,167, filed on Jan. 27, 2014, which is hereby incorporated by reference herein in its entirety.

### FIELD OF THE DISCLOSURE

The present disclosure relates to physiological signal processing, and more particularly relates to determining respiration information from regional oximetry signals obtained from a subject.

### SUMMARY

The present disclosure provides embodiments for a system comprising: an input for receiving a plurality of physiological signals responsive to total oxygen saturation in a region of a subject's tissue; and a processor configured to perform operations comprising: determining whether the plurality of physiological signals contain a reliable pulsatile component representing the subject's physiological pulse, and when it is determined that the reliable pulsatile component is present, determining respiration information based on the plurality of physiological signals and on the pulsatile component.

The present disclosure provides embodiments for a system comprising: an input for receiving a plurality of physiological signals responsive to total oxygen saturation in a region of a subject's tissue; and a processor configured to perform operations comprising: extracting a pulsatile component from at least two of the plurality of physiological signals by performing a cross-correlation operation; and determining respiration information based on the plurality of physiological signals and on the pulsatile component.

The present disclosure provides embodiments for a system comprising: an input for receiving two pairs of physiological signals, a first pair generated by a first optical detector located at a first location on a subject, and a second pair generated by a second optical detector located at a second location on the subject, the first pair responsive to emitted radiation at two distinct wavelengths and the second pair responsive to emitted radiation at two distinct wavelengths, wherein the first pair of physiological signals and the second pair of physiological signals are also responsive to oxygen saturation in a region of a subject's tissue through which the emitted radiation translates; and a processor configured for: extracting a baseline component from at least one of the physiological signals; and analyzing the baseline component to determine respiration information.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 is a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4 is a cross-sectional view of an illustrative regional oximeter sensor unit applied to a subject's tissue in accordance with some embodiments of the present disclosure;

FIG. 5 shows an illustrative light intensity signal that is modulated by respiration in accordance with some embodiments of the present disclosure;

FIG. 6 shows a comparison of portions of the illustrative light intensity signal of FIG. 5 in accordance with some embodiments of the present disclosure;

FIG. 7 shows an illustrative light intensity signal, a first derivative of the light intensity signal, and a second derivative of the light intensity signal in accordance with some embodiments of the present disclosure;

FIG. 8 shows illustrative steps for determining respiration information in accordance with some embodiments of the present disclosure; and

FIG. 9 shows illustrative steps for determining respiration information in accordance with some embodiments of the present disclosure; and

FIG. 10 shows illustrative steps for determining respiration information based on cross-correlation in accordance with some embodiments of the present disclosure.

### DETAILED DESCRIPTION OF THE FIGURES

The present disclosure is directed towards determining respiration information of a subject. In many cases, it is desirable to monitor the blood oxygen saturation in a region of a subject's tissue based on a plurality of physiological signals, such as one or more light intensity signals indicative of regional oxygen saturation. In some embodiments, light intensity signals indicative of regional oxygen saturation may also be indicative of pulsatile blood flow. Pulsatile blood flow may be dependent on a number of physiological functions such as cardiovascular function and respiration. For example, the light intensity signals indicative of regional oxygen saturation may exhibit a periodic component that generally corresponds to the heart beat of a patient. Respiration may also impact the pulsatile blood flow that is indicated by the light intensity signals indicative of regional oxygen saturation. It may thus be possible to calculate respiration information such as respiration rate or respiration effort from the amplitude and frequency modulation components of the light intensity signals indicative of regional oxygen saturation. However, in some instances, light intensity signals indicative of regional oxygen saturation may not be indicative of pulsatile blood flow, e.g. due to the location of the relevant signal detectors. In such instances, respiration may nevertheless impact the light intensity signals indicative of regional oxygen saturation. It may thus be possible to calculate respiration information such as respiration rate from the baseline components of the light intensity signals indicative of regional oxygen saturation. It may therefore be desirable to determine respiration information based on any of the amplitude, frequency, or baseline components of the light intensity signals indicative of regional oxygen saturation.

For purposes of clarity, the present disclosure is written in the context of the physiological signals being light intensity signals indicative of regional oxygen saturation generated by a regional oximeter. It will be understood that any other suitable physiological signal or any other suitable system may be used in accordance with the teachings of the present disclosure.

The foregoing techniques may be implemented in an oximeter. An oximeter is a medical device that may determine the oxygen saturation of an analyzed tissue. One common type of oximeter is a regional oximeter. A regional oximeter is used to estimate the blood oxygen saturation in a region of a subject's tissue. The regional oximeter may compute a differential absorption value for each of two or more wavelengths of light received at two different locations on the subject's body to estimate the regional blood oxygen saturation of hemoglobin in a region of the subject's tissue. For each wavelength of light, the regional oximeter may compare the amount of light absorbed by the subject's tissue in a first region to the amount of light absorbed by the subject's tissue in a second region to derive the differential absorption values. As opposed to pulse oximetry, which typically examines the oxygen saturation of pulsatile, arterial tissue, regional oximetry examines the oxygen saturation of blood in a region of tissue, which may include blood in the venous, arterial, and capillary systems. For example, a regional oximeter may include a sensor unit configured for placement on a subject's forehead and may be used to estimate the blood oxygen saturation of a region of tissue beneath the sensor unit (e.g., cerebral tissue).

In some embodiments, the oximeter may be a combined oximeter including a regional oximeter and a pulse oximeter. A pulse oximeter is a device for non-invasively measuring the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate, respiration rate, respiration effort, blood pressure, any other suitable physiological parameter, or any combination thereof. Pulse oximetry may be implemented using a photoplethysmograph.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine blood oxygen saturation using two wavelengths of light and a ratio-of-ratios calculation. In another example, the system may determine regional blood oxygen saturation using multiple wavelengths of light and a differential absorption technique. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, the regional oximeter may include a first sensor located at a first distance from the light source (e.g., the near detector) and a second sensor located at a second farther distance from the light source (e.g., the far detector). In some embodiments, the regional oximeter may include a near detector at a distance of 3 centimeters (cm) and a far detector at a distance of 4 cm from the light source, which may include, for example, one or more emitters. The distance between each detector and the light source affects the mean path length of the detected light and thus the depth of tissue through which the respective received wavelength of light passes. In other words, the light detected by the near detector may pass through shallow, superficial tissue, whereas the light detected by the far detector may pass through additional, deep tissue. In some embodiments, the regional oximeter's light source may include two or more emitters and one or more detectors. For example, a first emitter may be located a short distance from a detector, and the second emitter may be located a longer distance from the detector.

In some embodiments, multiple wavelengths of light may be received at both the near and far detectors, and the intensity of the multiple wavelengths of light may be computed and contrasted at each detector to derive regional blood oxygen saturation. For example, intensity signals for four wavelengths of light may be received at each of the near and far detectors, and the received intensity of each wavelength at the near detector may be subtracted from the received intensity of each wavelength at the far detector. The resulting light intensity signals may be indicative of, or responsive to, regional blood oxygen saturation because they may be used to compute the regional blood oxygen saturation of a region of deep tissue through which light received at the far detector passed. Because the far detector receives light that passes through deep tissue in addition to the shallow tissue through which the light passes and is received at the near detector, the regional saturation may be computed for just the deep tissue by subtracting out the intensity received by the near detector. For example, a regional oximeter on a subject's forehead may include near and far detectors spaced from the light source such that the near detector receives light that passes through the subject's forehead tissue, including the superficial skin, shallow tissue covering the skull, and the skull, and the far detector receives light that passes through the forehead tissue and brain tissue (i.e., cerebral tissue). In the example, the differences in the light intensities received by the near and far detectors may be used to derive an estimate of the regional blood oxygen saturation of the subject's cerebral tissue (i.e., cerebral blood oxygen saturation).

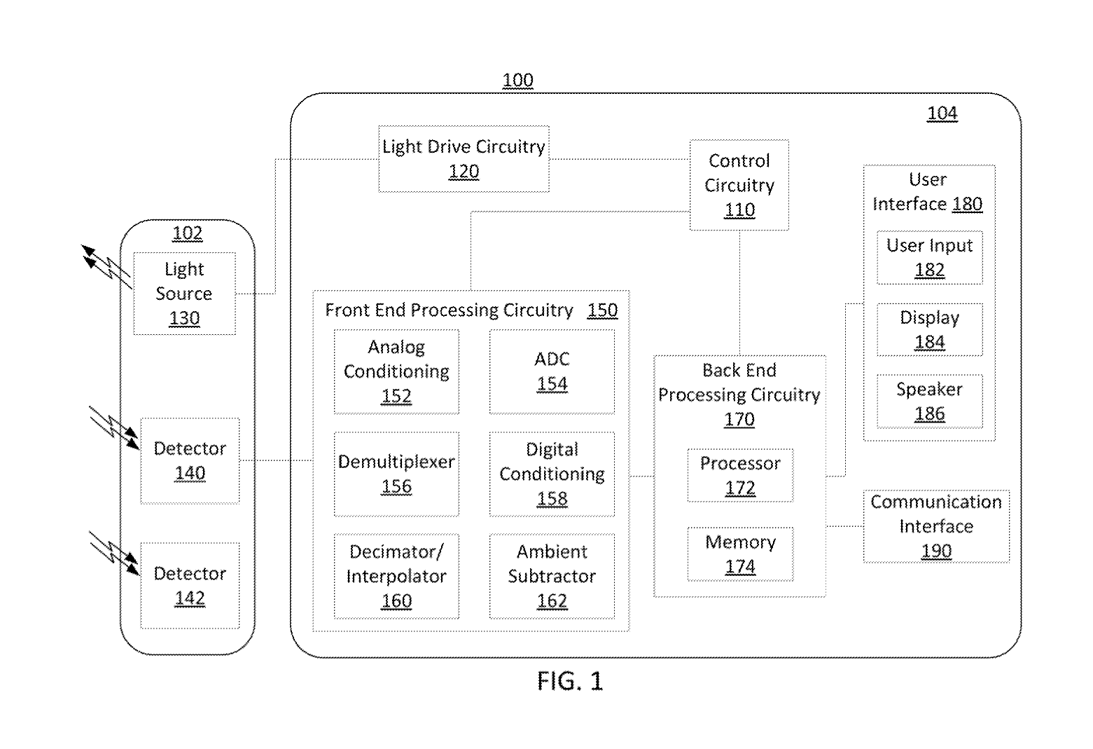

The following description and accompanying FIGS. 1-9 provide additional details and features of some embodiments of the present disclosure.

FIG. 1 is a block diagram of an illustrative physiological monitoring system 100 in accordance with some embodiments of the present disclosure. System 100 may include a sensor 102 and a monitor 104 for generating and processing physiological signals of a subject. In some embodiments, sensor 102 and monitor 104 may be part of an oximeter.

Sensor 102 of physiological monitoring system 100 may include light source 130, detector 140, and detector 142. Light source 130 may be configured to emit photonic signals having two or more wavelengths of light (e.g., red and IR) into a subject's tissue. For example, light source 130 may include a red light emitting light source and an IR light emitting light source, (e.g., red and IR light emitting diodes (LEDs)), for emitting light into the tissue of a subject to generate physiological signals. In one embodiment, the red wavelength may be between about 600 nm and about 700 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light. In some embodiments, light source 130 may be configured to emit two or more wavelengths of near-infrared light (e.g., wavelengths between 600 nm and 1000 nm) into a subject's tissue. In some embodiments, light source 130 may be configured to emit four wavelengths of light (e.g., 724 nm, 770 nm, 810 nm, and 850 nm) into a subject's tissue.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detectors 140 and 142 may be chosen to be specifically sensitive to the chosen targeted energy spectrum of light source 130.

In some embodiments, detectors 140 and 142 may be configured to detect the intensity of multiple wavelengths of near-infrared light. In some embodiments, detectors 140 and 142 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue, including skin, bone, and other shallow tissue (e.g., non-cerebral tissue and shallow cerebral tissue). Light may enter detector 142 after passing through the subject's tissue, including skin, bone, other shallow tissue (e.g., non-cerebral tissue and shallow cerebral tissue), and deep tissue (e.g., deep cerebral tissue). Detectors 140 and 142 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detectors 140 and 142. After converting the received light to an electrical signal, detectors 140 and 142 may send the detection signals to monitor 104, where the detection signals may be processed and physiological parameters may be determined (e.g., based on the absorption of the red and IR wavelengths in the subject's tissue at both detectors). In some embodiments, one or more of the detection signals may be preprocessed by sensor 102 before being transmitted to monitor 104.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate one or more light drive signals, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

Light drive circuitry 120, as discussed above, may be configured to generate a light drive signal that is provided to light source 130 of sensor 102. The light drive signal may, for example, control the intensity of light source 130 and the timing of when light source 130 is turned on and off. In some embodiments, light drive circuitry 130 provides one or more light drive signals to light source 130. Where light source 130 is configured to emit two or more wavelengths of light, the light drive signal may be configured to control the operation of each wavelength of light. The light drive signal may comprise a single signal or may comprise multiple signals (e.g., one signal for each wavelength of light).

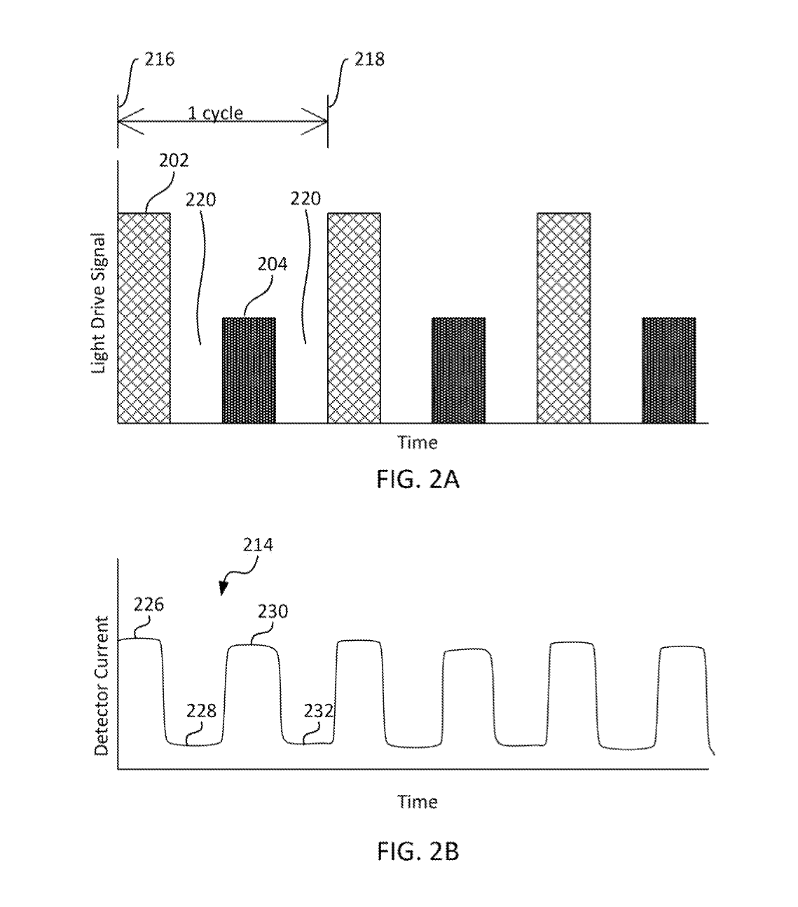

FIG. 2A shows an illustrative plot of a light drive signal including red light drive pulse 202 and IR light drive pulse 204 in accordance with some embodiments of the present disclosure. In the illustrated embodiment, light drive pulses 202 and 204 are shown as square waves. It will be understood that square waves are presented merely as an illustrative example, not by way of limitation, and that these pulses may include any other suitable signal, for example, shaped pulse waveforms, rather than a square waves. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130.

Red light drive pulse 202 may have a higher amplitude than IR light drive 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order to generate physiological signals that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof. It will also be understood that in systems that use more than two wavelengths of light, additional light drive pulses may be included in the light drive signal. For example, when four wavelengths of light are used, four light drive pulses, one for each wavelength of light, may be included in the light drive signal.

The light drive signal of FIG. 2A may also include “off” periods 220 between the red and IR light drive pulse. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive detection signals from detectors 140 and 142 and provide two or more processed signals to back end processing circuitry 170. The term “detection signals,” as used herein, may refer to any of the signals generated within front end processing circuitry 150 as it processes the output signal of detectors 140 and 142. Front end processing circuitry 150 may perform various analog and digital processing of the detector signals. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detectors 140 and 142 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valley 228 of detector current waveform 214 may be synchronous with periods of time during which no light is being emitted by the light source, or the light source is returning to dark, such as “off” period 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive detection signals, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signals. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof.

The conditioned analog signals may be processed by analog-to-digital converter 154, which may convert the conditioned analog signals into digital signals. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters. In some embodiments, analog-to-digital converter 154 may be a two channel analog-to-digital converter, where each channel is used for a respective detector waveform.

Demultiplexer 156 may operate on the analog or digital form of the detector signals to separate out different components of the signals. For example, detector current waveform 214 of FIG. 2B includes a red component corresponding to peak 226, an IR component corresponding to peak 230, and at least one ambient component corresponding to valley 228. Demultiplexer 156 may operate on detector current waveform 214 of FIG. 2B to generate a red signal, an IR signal, a first ambient signal (e.g., corresponding to the ambient component corresponding to valley 228 that occurs immediately after the peak 226), and a second ambient signal (e.g., corresponding to the ambient component corresponding to valley 232 that occurs immediately after the IR component 230). Demultiplexer 156 may operate under the control of control circuitry 110. For example, demultiplexer 156 may use timing control signals from control circuitry 110 to identify and separate out the different components of the detector signals.

Digital conditioning 158 may perform any suitable digital conditioning of the detector signals. Digital conditioning 158 may include any type of digital filtering of the signal (e.g., low pass, high pass, band pass, notch, averaging, or any other suitable filtering), amplifying, performing an operation on the signal, performing any other suitable digital conditioning, or any combination thereof.

Decimator/interpolator 160 may decrease the number of samples in the digital detector signals. For example, decimator/interpolator 160 may decrease the number of samples by removing samples from the detector signals or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying gain to the detection signals by analog conditioning 152 to map the expected range of the detection signals to the full or close to full output range of analog-to-digital converter 154. The output value of analog-to-digital converter 154, as a function of the total analog gain applied to each of the detection signals, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signals, thereby increasing the resolution of the converted signal. In some embodiments, the total analog gain may be reduced by a small amount so that small changes in the light levels incident on the detectors do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may result in fewer bits of resolution for the portion of the signal from the light source. This may have a detrimental effect on the signal-to-noise ratio of the detection signals. Accordingly, passive or active filtering or signal modification techniques may be employed to reduce the effect of ambient light on the detection signals that are applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further process physiological signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of regional oxygen saturation, blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. As is described herein, processor 172 may generate respiration morphology signals and determine respiration information from a physiological signal. Processor 172 may perform any suitable signal processing of a signal, such as any suitable band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store reference absorption curves, reference sets, calculated values, such as blood oxygen saturation, pulse rate, blood pressure, fiducial point locations or characteristics, initialization parameters, any other calculated values, or any combination thereof, in a memory device for later retrieval. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the subject, such as, for example, age ranges or medication families, which the user may select using user input 182. Additionally, display 184 may display, for example, one or more estimates of a subject's regional oxygen saturation generated by monitor 104 (referred to as an “rSO₂” measurement), an estimate of a subject's blood oxygen saturation generated by monitor 104 (referred to as an “SpO₂” measurement), pulse rate information, respiration rate information, respiration effort information, blood pressure, any other parameters, and any combination thereof. Display 184 may include any type of display such as a cathode ray tube display, a flat panel display such a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

The optical signal attenuated by the tissue of the subject can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., light intensity signals indicative of regional oxygen saturation) may involve operations that reduce the amount of noise present in the signals, control the amount of noise present in the signal, or otherwise identify noise components in order to prevent them from affecting measurements of physiological parameters derived from the sensor signals.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

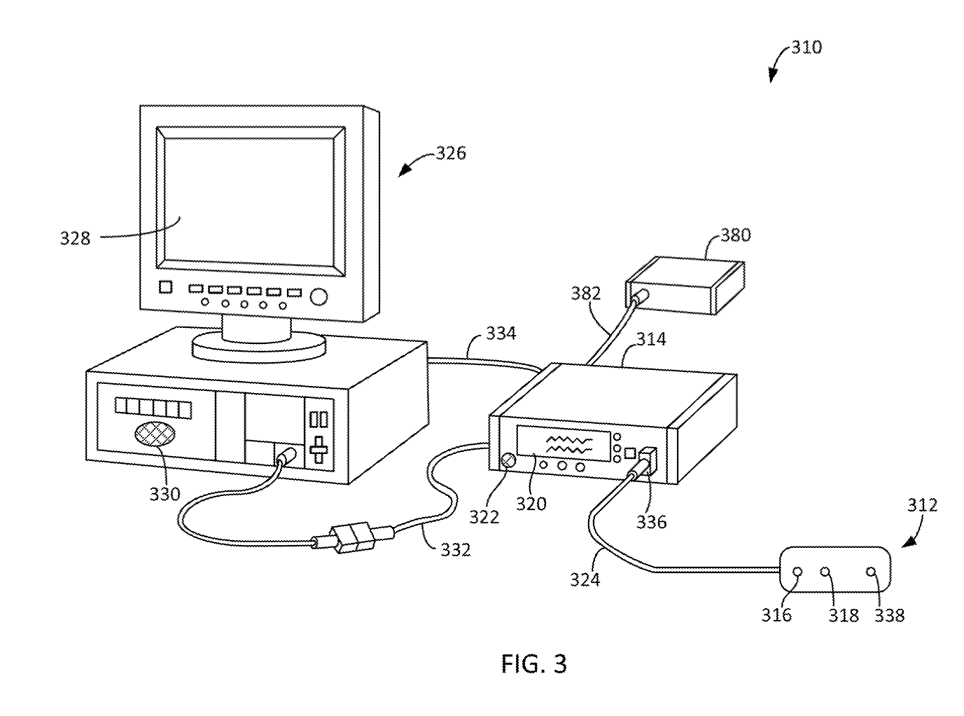

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. Detectors 318 and 338 may also be provided in sensor unit 312 for detecting the light that is reflected by or has traveled through the subject's tissue. Any suitable configuration of light source 316 and detectors 318 and 338 may be used. In some embodiments, sensor unit 312 may include multiple light sources and detectors, which may be spaced apart. In some embodiments, detector 318 (i.e., the near detector) may be positioned at a location closer to light source 316 than detector 338 (i.e., the far detector). Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and acoustic detection received from one or more sensor units such as sensor unit 312. For example, monitor 314 may be configured to determine regional oxygen saturation, pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as monitor 104 of FIG. 1.

In some embodiments, sensor unit 312 may be communicatively coupled to monitor 314 via a cable 324 at port 336. Cable 324 may include electronic conductors (e.g., wires for transmitting electronic signals from detectors 318 and 338), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's blood oxygen saturation and hemoglobin concentration, respiration rate, respiration effort, any other suitable parameters, or any combination thereof generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

As is described herein, monitor 314 may generate one or more light intensity signals based on the signal received from sensor unit 312. The light intensity signals may consist of data points that represent a pulsatile waveform. The pulsatile waveform may be modulated based on the respiration of a patient. Respiratory modulations may include baseline modulations, amplitude modulations, frequency modulations, respiratory sinus arrhythmia, any other suitable modulations, or any combination thereof. Respiratory modulations may exhibit different phases, amplitudes, or both, within a light intensity signal and may contribute to complex behavior (e.g., changes) of the light intensity signal. For example, the amplitude of the pulsatile waveform may be modulated based on respiration (amplitude modulation), the frequency of the pulsatile waveform may be modulated based on respiration (frequency modulation), and a signal baseline for the pulsatile waveform may be modulated based on respiration (baseline modulation). Monitor 314 may analyze the light intensity signals(e.g., by generating respiration morphology signals from the light intensity signals, generating a combined autocorrelation sequence based on the respiration morphology signals, and calculating respiration information from the combined autocorrelation sequence) to determine respiration information based on one or more of these modulations of the light intensity signal.

As is described herein, respiration information may be determined from the light intensity signals generated by monitor 314. However, it will be understood that the light intensity signal could be transmitted to any suitable device for the determination of respiration information, such as a local computer, a remote computer, a nurse station, mobile devices, tablet computers, or any other device capable of sending and receiving data and performing processing operations. Information may be transmitted from monitor 314 in any suitable manner, including wireless (e.g., WiFi, Bluetooth, etc.), wired (e.g., USB, Ethernet, etc.), or application-specific connections. The receiving device may determine respiration information as described herein.

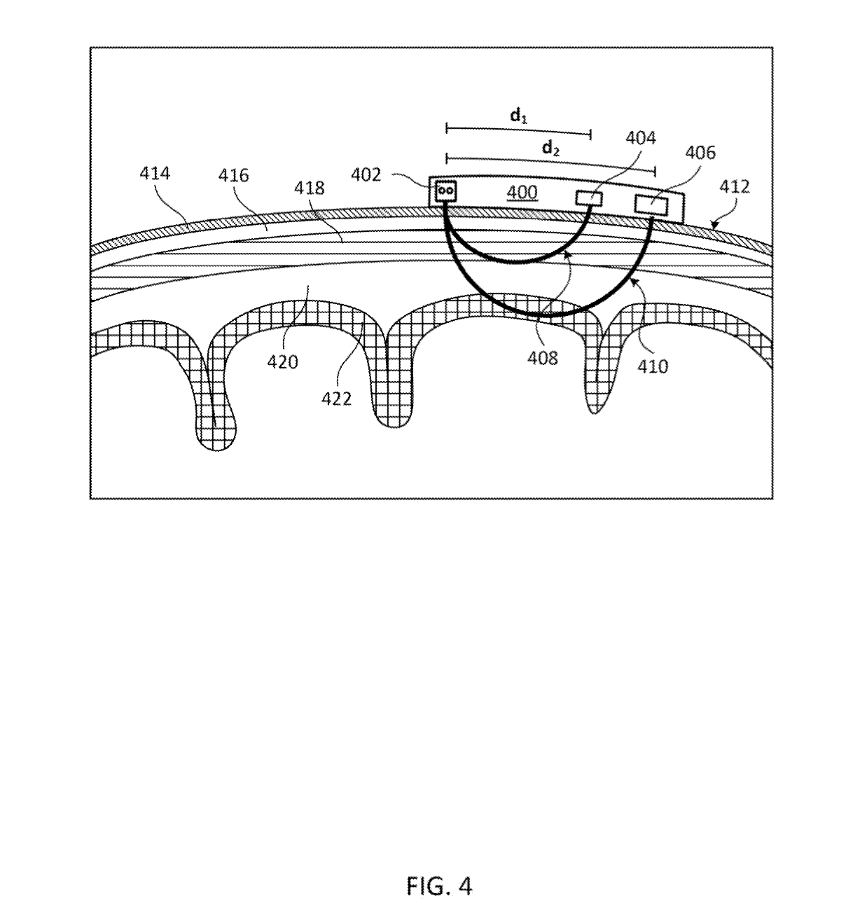

In some embodiments, any of the processing components and/or circuits, or portions thereof, of FIGS. 1 and 3, including sensors 102 and 312 and monitors 104, 314, and 326 may be referred to collectively as processing equipment. For example, processing equipment may be configured to amplify, filter, sample and digitize an input signal from sensor 102 or 312 (e.g., using an analog-to-digital converter), calculate physiological information and metrics from the digitized signal, and display a trace of the physiological information. In some embodiments, all or some of the components of the processing equipment may be referred to as a processing module. In some embodiments, the processing equipment may be part of a regional oximetry system, and sensors 102 and 312 of FIGS. 1 and 3 may correspond to regional oximeter sensor unit 400 of FIG. 4, described below.

FIG. 4 is a cross-sectional view of an illustrative regional oximeter sensor unit 400 applied to a subject's cranium in accordance with some embodiments of the present disclosure. Regional oximeter sensor unit 400 includes light source 402, near detector 404, and far detector 406 and is shown as positioned on a subject's forehead 412. In the illustrated embodiment, light source 402 generates a light signal, which is shown traveling first and second mean path lengths 408 and 410, which traverse the subject's cranial structure at different depths. The subject's cranial structure includes outer skin 414, shallow tissue 416, and cranial bone 418 (i.e., the frontal shell of the skull). Beneath cranial bone 418 is Dura Mater 420 and cerebral tissue 422.

In some embodiments, light source 402 of sensor unit 400 may include one or more emitters for emitting light into the tissue of a subject to generate physiological signals. Detectors 404 and 406 may be positioned on sensor unit 400 such that near detector 404 is located at a distance d₁ from light source 402 and far detector 406 is located at a distance d₂ from light source 402. As shown, distance d₁ is shorter than distance d₂, and it will be understood that any suitable distances d₁ and d₂ may be used such that mean path length 408 of light detected by near detector 404 is shorter than the mean path length 410 of far detector 406. Near detector 404 may receive the light signal after it has traveled first mean path length 408, and far detector 406 may receive the light signal after it has traveled second mean path length 410. First mean path length 408 may traverse the subject's outer skin 414, shallow tissue 416, cranial bone 418, and Dura Mater 420. In some embodiments, first mean path length 408 may also traverse shallow cerebral tissue 422. Second mean path length 410 may traverse the subject's outer skin 414, shallow tissue 416, cranial bone 418, Dura Mater 420, and cerebral tissue 422.

In some embodiments, regional oximeter sensor unit 400 may be part of a regional oximetry system for determining the amount of light absorbed by a region of a subject's tissue. As described in detail above, for each wavelength of light, an absorption value may be determined based on the light signal on first mean path length 408 received at near detector 404, and an absorption value may be determined based on the light signal on second mean path length 410 received at far detector 406. For each wavelength of light, a differential absorption value may be computed based on the difference between the absorption values determined for near detector 404 and far detector 406. The differential absorption values may be representative of the amount of light absorbed by cerebral tissue 422 at each wavelength. In some embodiments, the differential absorption values ΔA_{λi,j} may be given by:

ΔA_{λi,j}=A_{λi}−A_{λj}, (1)

where A_{λi} denotes the attenuation of light between light source 402 and far detector 406, A_{λj} denotes the attenuation of light between light source 402 and near detector 404, and the λ denotes a wavelength of light. In some embodiments, a detected light signal may be normalized based on the amount of light emitted by light source 402 and the amount of light detected at the respective detector (i.e., near detector 404 or far detector 406). The processing equipment may determine the differential absorption values ΔA_{λi,j} based on eq. 1, using normalized values for the attenuation of light between light source 402 and far detector 406 and the attenuation of light between light source 402 and near detector 404. Once the differential absorption values ΔA_{λi,j} are determined, the regional blood oxygen saturation can be determined or estimated using any suitable technique for relating the regional blood oxygen saturation to the differential absorption values ΔA_{λi,j}.

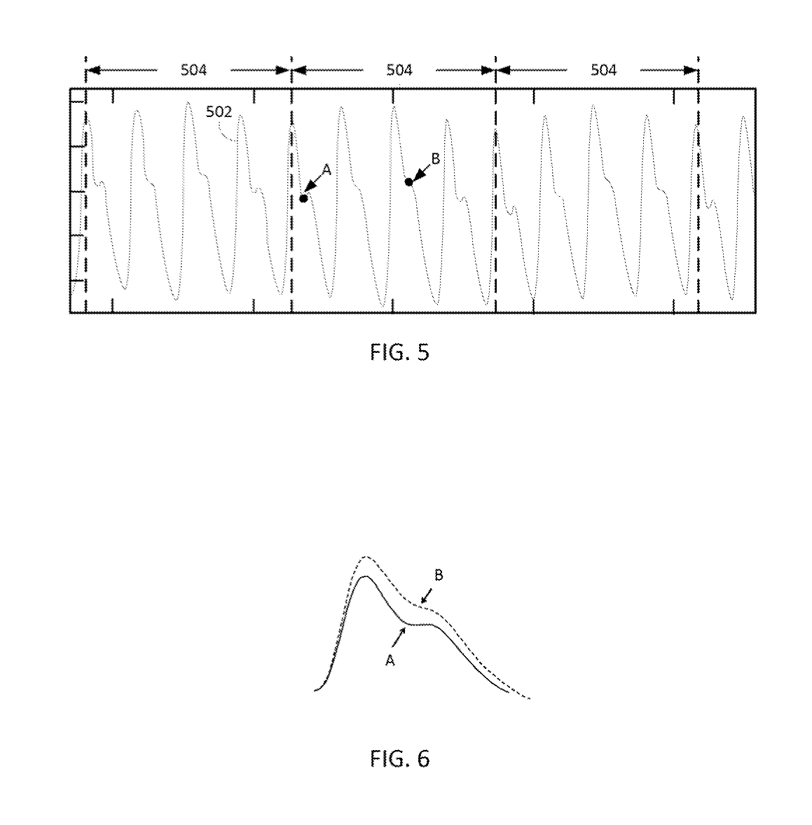

In some embodiments, physiological signals used to determine blood oxygen saturation may be indicative of pulsatile blood flow, and may thus exhibit pulsatile components. For example, a PPG signal received by a pulse oximeter may contain a pulsatile component. In some instances, one or more light intensity signals indicative of regional oxygen saturation received by a regional oximeter may also contain pulsatile components. For example, regional oximeters measuring a particular region of a subject's tissue may receive light intensity signals indicative of regional oxygen saturation that contain pulsatile components if the particular region being monitored is located such that pulsatile blood flow impacts the light intensity signals indicative of regional oxygen saturation. As will be discussed in detail below with reference to FIGS. 5-7, a number of morphology metrics related to respiration may be derived from these light intensity signals indicative of regional oxygen saturation which contain pulsatile components.

FIG. 5 shows an illustrative light intensity signal 502 that is modulated by respiration in accordance with some embodiments of the present disclosure. light intensity signal 502 may be a periodic signal that is indicative of changes in pulsatile blood flow. Each cycle of light intensity signal 502 may generally correspond to a pulse, such that a heart rate may be determined based on light intensity signal 502. Each respiratory cycle 504 may correspond to a breath. The period of a respiratory cycle may typically be longer than the period of a pulsatile cycle, such that any changes in the pulsatile blood flow due to respiration occur over a number of pulsatile cycles. The volume of the pulsatile blood flow may also vary in a periodic manner based on respiration, resulting in modulations to the pulsatile blood flow such as amplitude modulation, frequency modulation, and baseline modulation. This modulation of light intensity signal 502 due to respiration may result in changes to the morphology of light intensity signal 502.

FIG. 6 shows a comparison of portions of the illustrative light intensity signal 502 of FIG. 5 in accordance with some embodiments of the present disclosure. The signal portions compared in FIG. 6 may demonstrate differing morphology due to respiration modulation based on the relative location of the signal portions within a respiratory cycle 504. For example, a first pulse associated with the respiratory cycle may have a relatively low amplitude (indicative of amplitude and baseline modulation) as well as an obvious distinct dichrotic notch as indicated by point A. A second pulse may have a relatively high amplitude (indicative of amplitude and baseline modulation) as well as a dichrotic notch that has been washed out as depicted by point B. Frequency modulation may be evident based on the relative period of the first pulse and second pulse. Referring again to FIG. 5, by the end of the respiratory cycle 504 the pulse features may again be similar to the morphology of A. Although the impact of respiration modulation on the morphology of a particular light intensity signal 502 has been described herein, it will be understood that respiration may have varied effects on the morphology of a light intensity signal other than those depicted in FIGS. 5 and 6.

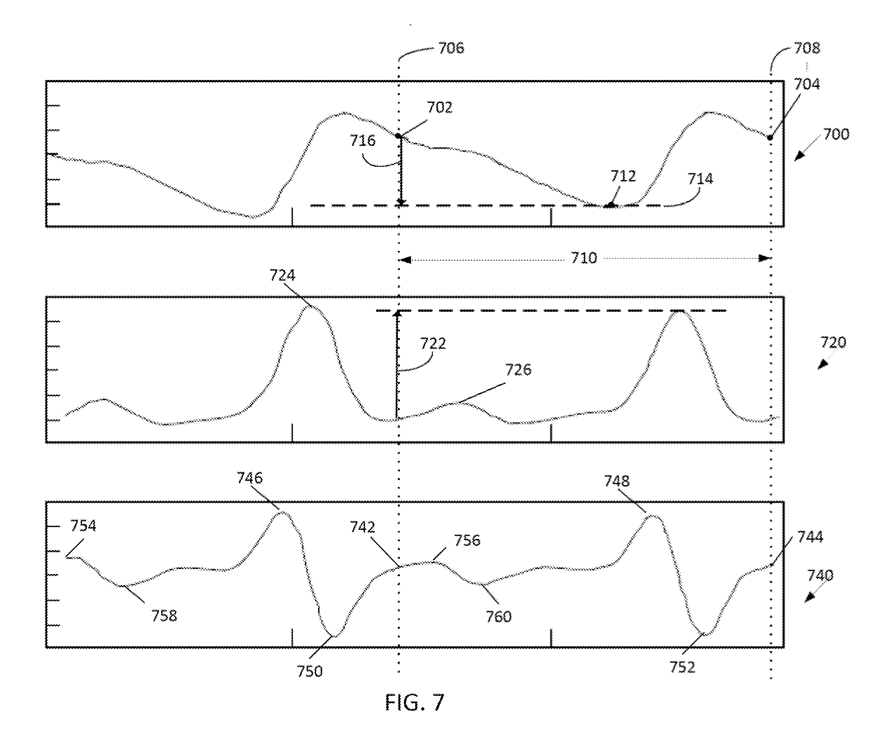

FIG. 7 depicts exemplary signals used for calculating morphology metrics from a received light intensity signal. The abscissa of each plot of FIG. 7 may represent time and the ordinate of each plot may represent magnitude. Light intensity signal 700 may be a received light intensity signal, first derivative signal 720 may be a signal representing the first derivative of the light intensity signal 700, and second derivative signal 740 may be a signal representing the second derivative of the light intensity signal 700. As will be described herein, morphology metrics may be calculated for portions of these signals, and a series of morphology metric calculations calculated over time may be processed to generate the respiration morphology signals. Although particular morphology metric calculations are set forth below, each of the morphology metric calculations may be modified in any suitable manner.

Although morphology metrics may be calculated based on any suitable portions of the light intensity signal 700 (as well as the first derivative signal 720, second derivative signal 740, and any other suitable signals that may be generated from the light intensity signal 700), in an exemplary embodiment, morphology metrics may be calculated for each fiducial-defined portion such as fiducial defined portion 710 of the light intensity signal 700. Exemplary fiducial points 702 and 704 are depicted for light intensity signal 700, and fiducial lines 706 and 708 demonstrate the location of fiducial points 702 and 704 relative to first derivative signal 720 and second derivative signal 740.

Although it will be understood that fiducial points may be identified in any suitable manner, in exemplary embodiments fiducial points may be identified based on features of the light intensity signal 720 or any derivatives thereof (e.g., first derivative signal 720 and second derivative signal 740) such as peaks, troughs, points of maximum slope, dichrotic notch locations, pre-determined offsets, any other suitable features, or any combination thereof. Fiducial points 702 and 704 may define a fiducial-defined portion 710 of light intensity signal 700. The fiducial points 702 and 704 may define starting and ending points for determining morphology metrics, and the fiducial-defined portion 710 may define a relevant portion of data for determining morphology metrics. It will be understood that other starting points, ending points, and relative portions of data may be utilized to determine morphology metrics.

An exemplary morphology metric may be a down metric. The down metric is the difference between a first (e.g., fiducial) sample of a fiducial-defined portion (e.g., fiducial defined portion 710) of the light intensity signal (e.g., light intensity signal 700) and a minimum sample (e.g., minimum sample 712) of the fiducial-defined portion 710 of the light intensity signal 700. The down metric may also be calculated based on other points of a fiducial-defined portion. The down metric is indicative of physiological characteristics which are related to respiration, e.g., amplitude and baseline modulations of the light intensity signal. In an exemplary embodiment, fiducial point 702 defines the first location for calculation of a down metric for fiducial-defined portion 710. In the exemplary embodiment, the minimum sample of fiducial-defined portion 710 is minimum point 712, and is indicated by horizontal line 714. The down metric may be calculated by subtracting the value of minimum point 712 from the value of fiducial point 702, and is depicted as down metric 716.

Another exemplary morphology metric may be a kurtosis metric for a fiducial-defined portion. Kurtosis measures the peakedness of the light intensity signal 700 or a derivative thereof (e.g., first derivative signal 720 or second derivative signal 740). In an exemplary embodiment, the kurtosis metric may be based on the peakedness of the first derivative signal 720. The peakedness is sensitive to both amplitude and period (frequency) changes, and may be utilized as an input to generate respiration morphology signals that may be used to determine respiration information such as respiration rate. Kurtosis may be calculated based on the following formulae:

$$D = \frac{1}{n} {\sum }_{i = 1}^{n} {( {x}_{i}^{′} - \overline{{x}^{′}} )}^{2}$$

$$Kurtosis = \frac{1}{{nD}^{2}} {\sum }_{i = 1}^{n} {( {x}_{i}^{′} - \overline{{x}^{′}} )}^{4}$$

where:
xᵢ′=ith sample of 1^{st} derivative;
x̅′̅=mean of 1st derivative of fiducial-defined portion;
n=set of all samples in the fiducial-defined portion

Another exemplary morphology metric may be a delta of the second derivative (DSD) between consecutive fiducial-defined portions, e.g., at consecutive fiducial points. Measurement points 742 and 744 for a DSD calculation are depicted at fiducial points 702 and 704 as indicated by fiducial lines 706 and 708. The second derivative signal is indicative of the curvature of a signal. Changes in the curvature of the light intensity signal 700 that can be identified with second derivative signal 740 are indicative of changes in internal pressure that occur during respiration, particularly changes near the peak of a pulse. By providing a metric of changes in curvature of the light intensity signal, the DSD morphology metric may be utilized as an input to determine respiration information, such as respiration rate. The DSD metric may be calculated for each fiducial-defined portion by identifying the value of the second derivative signal 740 at the current fiducial point (e.g., fiducial point 742 of fiducial-defined portion 710) and subtracting from that the value of the second derivative signal 740 at the next fiducial point (e.g., fiducial point 744 of fiducial-defined portion 710).

Another exemplary morphology metric may be an up metric measuring the up stroke of the first derivative signal 720 of the light intensity signal. The up stroke may be based on an initial starting sample (fiducial point) and a maximum sample for the fiducial-defined portion and is depicted as up metric 722 for a fiducial point corresponding to fiducial line 706. The up metric may be indicative of amplitude and baseline modulation of the light intensity signal, which may be related to respiration information as described herein. Although an up metric is described herein with respect to the first derivate signal 720, it will be understood that an up metric may also be calculated for the light intensity signal 700 and second derivative signal 740.

Another exemplary morphology metric may be a skew metric measuring the skewness of the original light intensity signal 700 or first derivative 720. The skew metric is indicative of how tilted a signal is, and increases as the light intensity signal is compressed (indicating frequency changes in respiration) or the amplitude is increased. The skewness metric is indicative of amplitude and frequency modulation of the light intensity signal, which may be related to respiration information as described herein. Skewness may be calculated as follows:

$$g 1 = \frac{{m}_{3}}{{m}_{2}^{3 \text{/} 2}} = \frac{\frac{1}{n} {{Σ}_{i = 1}^{n} ( {x}_{i} - \overline{x} )}^{3}}{{( \frac{1}{n} {{Σ}_{i = 1}^{n} ( {x}_{i} - \overline{x} )}^{2} )}^{3 \text{/} 2}}$$

where:
xᵢ=ith sample;
x̅=mean of the samples of the fiducial-defined portion;
m₃=third moment;
m₂=second moment; and
n=total number of samples.

Another exemplary morphology metric may be a b/a ratio metric (i.e., b/a), which is based on the ratio between the a-peak and b-peak of the second derivative signal 740. Light intensity signal 700, first derivative signal 720, and second derivative signal 700 may include a number of peaks (e.g., four peaks corresponding to maxima and minima) which may be described as the a-peak, b-peak, c-peak, and d-peak, with the a-peak and c-peak generally corresponding to local maxima within a fiducial defined portion and the b-peak and d-peak generally corresponding to local minima within a fiducial defined portion. For example, the second derivative of the light intensity signal may include four peaks: the a-peak, b-peak, c-peak, and d-peak. Each peak may be indicative of a respective systolic wave, i.e., the a-wave, b-wave, c-wave, and d-wave. On the depicted portion of the second derivative of the light intensity signal 740, the a-peaks are indicated by points 746 and 748, the b-peaks by points 750 and 752, the c-peaks by points 754 and 756, and the d-peaks by points 758 and 760. The b/a ratio measures the ratio of the b-peak (e.g., 750 or 752) and the a-peak (e.g., 746 or 748). The b/a ratio metric may be indicative of the curvature of the light intensity signal, which demonstrates frequency modulation based on respiration information such as respiration rate. The b/a ratio may also be calculated based on the a-peak and b-peak in higher order signals such as light intensity signal and first derivative light intensity signal 720.

Another exemplary morphology metric may be a c/a ratio (i.e., c/a), which is calculated from the a-peak and c-peak of a signal. For example, first derivate light intensity signal 720 may have a c-peak 726 which corresponds to the maximum slope near the dichrotic notch of light intensity signal 700, and an a-peak 724 which corresponds to the maximum slope of the light intensity signal 700. The c/a ratio of the first derivative is indicative of frequency modulation of the light intensity signal, which is related to respiration information such as respiration rate as described herein. A c/a ratio may be calculated in a similar manner for light intensity signal 700 and second derivative signal 740.

Another exemplary morphology metric may be a i_b metric measuring the time between two consecutive local minimum (b) locations 750 and 752 in the second derivative 740. The i_b metric is indicative of frequency modulation of the light intensity signal, which is related to respiration information such as respiration rate as described herein. The i_b metric may also be calculated for light intensity signal 700 or first derivative signal 720.

Another exemplary morphology metric may be a peak amplitude metric measuring the amplitude of the peak of the original light intensity signal 700 or of the higher order derivatives 720 and 740. The peak amplitude metric is indicative of amplitude modulation of the light intensity signal, which is related to respiration information such as respiration rate as described herein.

Another exemplary morphology metric may be a center of gravity metric measuring the center of gravity of a fiducial-defined portion from the light intensity signal 700 in either or both of the x and y coordinates. The center of gravity is calculated as follows:

Center of gravity (x)=Σ(xᵢ*yᵢ)/Σyᵢ

Center of gravity (y)=Σ(xᵢ*yᵢ)/Σxᵢ

The center of gravity metric of the x coordinate for a fiducial-defined portion is indicative of frequency modulation of the light intensity signal, which is related to respiration information such as respiration rate as described herein. The center of gravity metric of the y coordinate for a fiducial-defined portion is indicative of amplitude modulation of the light intensity signal, which is related to respiration information such as respiration rate as described herein.

Another exemplary morphology metric is an area metric measuring the total area under the curve for a fiducial-defined portion of the light intensity signal 700. The area metric is indicative of frequency and amplitude modulation of the light intensity signal, which is related to respiration information such as respiration rate as described herein.

Another morphology metric is the light intensity amplitude metric. This metric represents the amplitude of the patient's light intensity signal. In some embodiments, the light intensity amplitude metric is normalized to the baseline (i.e., DC component) of the underlying light intensity signal.

Another morphology metric is the light intensity amplitude modulation metric. This metric represents the modulation of amplitude over time on a patient's light intensity signal.

Another morphology metric is the frequency modulation metric. This metric represents the modulation of periods between fiducial points on a physiological signal, such as a light intensity signal.

Although a number of morphology metrics have been described herein, it will be understood that other morphology metrics may be calculated from light intensity signal 700, first derivative signal 720, second derivative signal 740, and any other order of the light intensity signal. It will also be understood that any of the morphology metrics described above may be modified to capture aspects of respiration information or other physiological information that may be determined from a light intensity signal.

In some embodiments, each series of morphology metric values may be further processed in any suitable manner to generate the respiration morphology signals. Although any suitable processing operations may be performed for each series of morphology metric values, in an exemplary embodiment, each series of morphology metric values may be filtered (e.g., based on frequencies associated with respiration) and interpolated to generate the plurality of respiration morphology signals.

In an embodiment, an autocorrelation sequence may be generated for each of the respiration morphology signals. The peaks of an autocorrelation correspond to portions of the signal that include the same or similar information. Thus, the peaks of the autocorrelation sequences may correspond to periodic aspects of the underlying respiration morphology signals, which in turn may correspond to respiration information such as respiration rate.

Although it will be understood that respiration information such as respiration rate may be determined from one or more of the autocorrelation sequences in any suitable manner, in an embodiment, the autocorrelation sequences may be combined to generate a combined autocorrelation sequence and the respiration rate may be determined based on a lag (i.e., time delay associated with the period of breathing) associated with a peak of the autocorrelation sequence. Although the autocorrelation sequences may be combined in any suitable manner, in an exemplary embodiment the autocorrelation sequences having the most periodic information may be given the greatest weight in the combination.

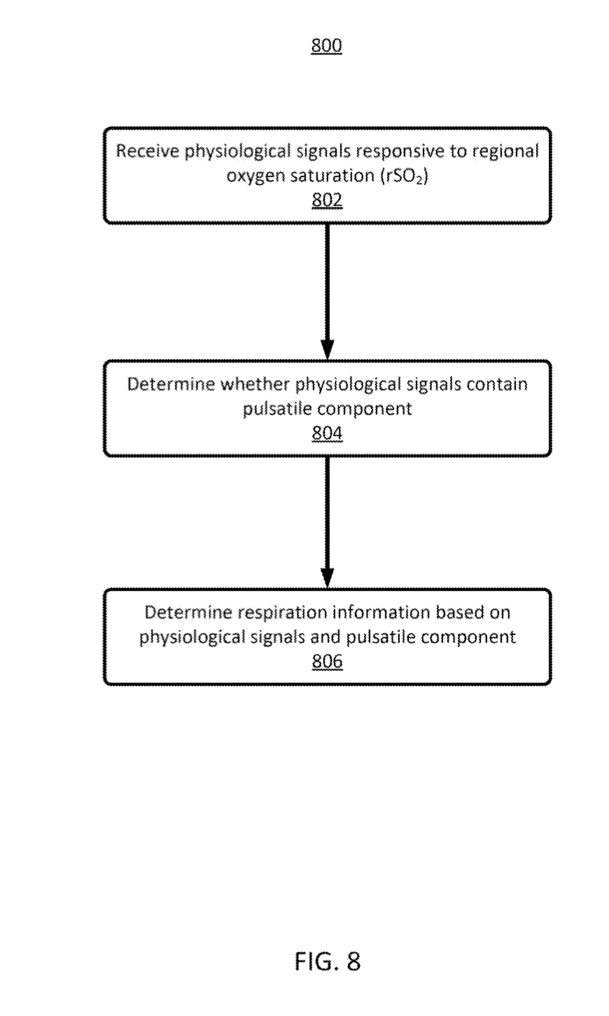

FIG. 8 shows illustrative steps for determining respiration information from a plurality of physiological signals in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310.

At step 802, monitoring system 310 may receive physiological signals responsive to, or indicative of, regional oxygen saturation of a subject's tissue. In an embodiment, the physiological signals received may include a plurality of light intensity or absorption signals generated by a regional oximeter as described herein. For example, the physiological signals may include light intensity signals received from separate detectors placed at different locations in relation to the subject. In some embodiments, each of the physiological signals may correspond to measured intensity of different wavelengths of light. In some embodiments, each of the physiological signals may correspond to a differential absorption value for each of two or more wavelengths of light received at two different locations on the subject's body. Although the physiological signals may be processed in any suitable manner, in an embodiment, the physiological signals may be analyzed each 5 seconds, and for each 5 second analysis window, the most recent 45 seconds of the physiological signal may be analyzed.

At step 804 monitoring system 310 may determine whether the physiological signals contain a pulsatile component representing the subject's pulse. Although the physiological signals may be processed in any suitable manner to determine whether any of the physiological signals contain a reliable pulsatile component, in some embodiments, monitoring system 310 may process the physiological signals using time-frequency analysis. For example, monitoring system 310 may apply Short-time Fourier transform or Wavelet transform techniques to any of the physiological signals to determine if the signals exhibit periodic components corresponding to the subject's heartbeat. In some embodiments, monitoring system 310 may process the physiological signals using time domain analysis. For example, monitoring system 310 may apply autocorrelation techniques to any of the physiological signals to determine if the signals exhibit periodic components corresponding to the subject's heartbeat. If it is determined at step 804 that a pulsatile component is present, the system may proceed to step 806.

In another step (not shown) monitoring system 310 may determine whether the pulsatile component is a reliable pulsatile component. In some embodiments, monitoring system 310 may calculate confidence values associated with each of the physiological signals that are indicative of the reliability of the pulsatile component detected in the physiological signals and compare these confidence values to a threshold confidence value. In some instances, monitoring system 310 may determine whether any of the physiological signals contain a reliable pulsatile component based on the comparison of the confidence value to the threshold confidence value. In some embodiments, neural networks may be utilized to determine whether the pulsatile component is a reliable pulsatile component. For example, inputs to the network may include any suitable metrics derived from the pulsatile component being analyzed, metrics derived from pulsatile components previously determined to be reliable, or any suitable combination therof. For example, sharp up-slopes that are characteristic of the pulse may be identified by analyzing the skew of the derivative of the current pulsatile component and comparing it to the skew of the derivative of a previous pulsatile component that was determined to be reliable. In some instances, the neural networks may be trained using historical data including known heart rates and pulse periods. In some instances, the neural network may output a number between 0 and 1 indicating the reliability of the pulse, where a value of 1 indicates the highest reliability. If it is determined at step 804 that the reliable pulsatile component is present, the system may proceed to step 806.

At step 806, monitoring system 310 may determine respiration information based on the plurality of physiological signals and on the pulsatile component. In some embodiments, one or more respiration morphology signals may be generated from the physiological signals, such as a down respiration morphology signal, a DSD respiration morphology signal, a kurtosis respiration morphology signal, any of the respiration morphology signals described herein, and any other suitable respiration morphology signal. Although a respiration morphology signal may be generated in any suitable manner, in an embodiment, each respiration morphology signal may be generated based on calculating a series of morphology metrics from one or more physiological signals. One or more morphology metrics maybe calculated for each portion of the physiological signal (e.g., for each fiducial defined portion), a series of morphology metrics may be calculated over time, and the series of morphology metrics may be processed to generate one or more morphology metric signals. In some embodiments, an autocorrelation sequence may be generated for each of the respiration morphology signals and respiration information may be determined based on peaks of the autocorrelation sequences which correspond to periodic aspects of the underlying respiration signals. In some instances, the autocorrelation sequences may be combined to generate a combined autocorrelation sequence and the respiration information may be determined based on a lag (i.e., time delay associated with the period of breathing) associated with a peak of the autocorrelation sequence.

In some embodiments, separate respiration morphology signals and autocorrelation sequences may be generated for each of the plurality of physiological signals generated by the regional oximeter. In some instances, each of the plurality of physiological signals generated by the regional oximeter may have a confidence value associated with it based on any suitable method. For example, the confidence value associated with a physiological signal may be determined based on the amount of the filtering that was required to remove unwanted portions of the signal during pre-processing. Monitoring system 310 may select the physiological signal with the highest confidence value, and determine respiration information based on the respiration morphology signals and autocorrelation sequences corresponding to that physiological signal.

In some embodiments, at least two of the physiological signals generated by the regional oximeter may be combined to generate a combined signal. Although any suitable method for combining signals may be used, in some instances, the physiological signals may be averaged to generate a combined signal. In some embodiments, respiration morphology signals and autocorrelation sequences may be generated based on the combined signal, and respiration information may be determined based thereon.

In some embodiments, the respiration information that may be determined by monitoring system 310 is respiration rate. Although respiration rate may be determined by any suitable method, in some instances monitoring system 310 may determine respiration rate by determining a period P associated with the respiration morphology signals and/or autocorrelation sequences, and determining respiration rate RR, by the equation RR=60/P, where P is the period determined in seconds, and RR is the respiration rate in units of breath per minute.

In some embodiments, the respiration information that may be determined by monitoring system 310 is respiration effort. Although respiration rate may be determined by any suitable method, in some instances monitoring system 310 may determine respiration rate by determining an amplitude associated with the respiration morphology signals and/or autocorrelation sequences, and determining respiration effort based thereon.

In an additional step (not illustrated), monitoring system 310 may determine a value indicative of oxygen saturation in a region of the subject's tissue (e.g., rSO₂) based on the physiological signals. In some embodiments, monitoring system 310 may determine rSO₂ by determining differential absorption values and using any suitable technique for relating the regional blood oxygen saturation to the differential absorption values.

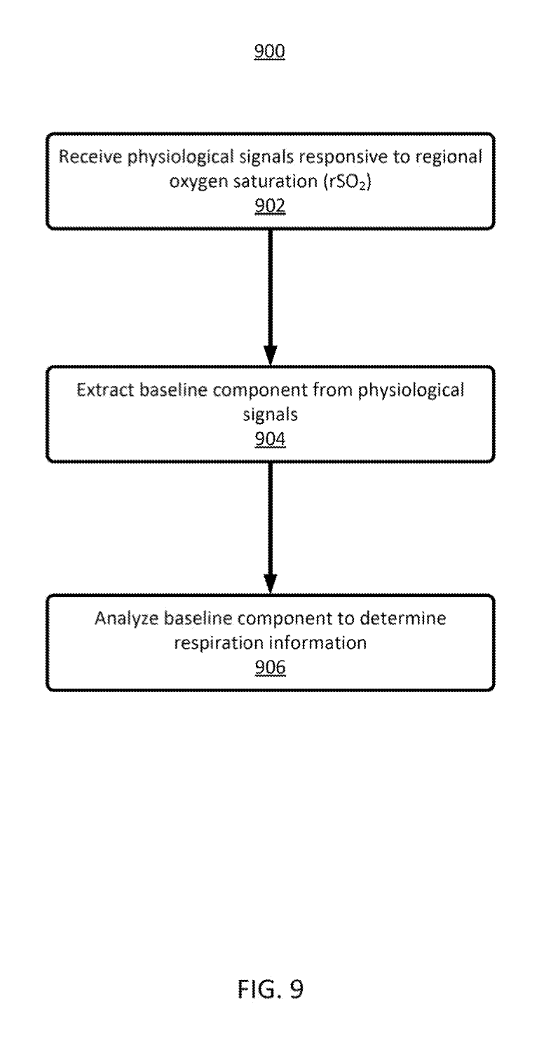

FIG. 9 shows illustrative steps for determining respiration information from a plurality of physiological signals in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310.

At step 902, monitoring system 310 may receive physiological signals responsive to, or indicative of, regional oxygen saturation of a subject's tissue. Monitoring system 310 may receive any of the physiological signals described above with respect to step 802, including a plurality of light intensity or absorption signals generated by a regional oximeter, light intensity signals received from separate detectors placed at different locations in relation to the subject, and signals corresponding to measured intensity of different wavelengths of light. In some embodiments, monitoring system 310 may receive two pairs of physiological signals, where each pair is generated by separate detectors located at different locations on the subject. In some instances, each pair comprises two signals responsive to two distinct wavelengths of light.

At step 904, monitoring system 310 may extract one or more baseline components from any one or more of the physiological signals. Although any suitable methods may be used to extract a baseline component from the physiological signals, in some embodiments, a baseline component may be acquired from the physiological signals based on sampling of the signals and identifying modulations of the physiological signals that are not the result of amplitude modulation (i.e., that are due to the changing DC portion of the signal rather than an increase in the peak-to-peak strength of the signal). In some embodiments, any one or more filtering techniques may be used on any one or more of the physiological signals to extract a baseline component. For example, a high pass filter, a low pass filter, a band-pass filter, a band-stop filter, any other suitable filter, or any combination thereof may be used by implementing any suitable cut-off frequencies relevant to respiration. In some embodiments, a baseline component may be extracted by the use of function fitting techniques. For example, a polynomial or other suitable function may be fit to any one or more of the physiological signals to extract the baseline modulations of any of the physiological signals. In some embodiments, monitoring system 310 may use wavelet analysis to determine baseline component. For example, the system may perform a wavelet transform on any one or more of the physiological signals, generate a scalogram, and extract baseline information based on modulations identified in bands of the scalogram.

At step 906, monitoring system 310 may analyze the baseline component to determine respiration information. Although any suitable methods may be used to analyze the baseline component to determine respiration information, in some embodiments, an autocorrelation may be performed on the baseline component. The peaks of an autocorrelation correspond to portions of the signal that include the same or similar information. Thus, the peaks of the autocorrelation signal may correspond to periodic aspects of the baseline component. In some embodiments, respiration information such as respiration rate or respiration effort can then be determined from the autocorrelation signal in the same way as described above with respect to step 806. In some embodiments, if the baseline component was extracted using wavelet transforms and scalograms, respiration information may be determined by analysis of modulations in a breathing band of a scalogram.

As described above with respect to method 800, monitoring system 310 may perform the additional step of determining a value indicative of oxygen saturation in a region of the subject's tissue (e.g., rSO₂) based on the physiological signals.

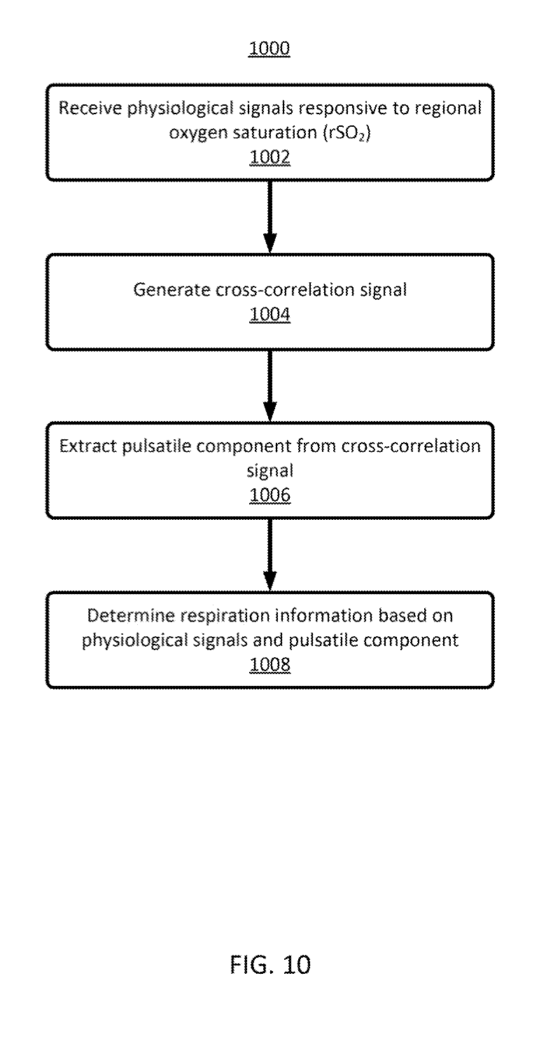

FIG. 10 shows illustrative steps for determining respiration information from a plurality of physiological signals in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310.

At step 1002, monitoring system 310 may receive physiological signals responsive to, or indicative of, regional oxygen saturation of a subject's tissue. Monitoring system 310 may receive any of the physiological signals described above with respect to step 802, including a plurality of light intensity or absorption signals generated by a regional oximeter, light intensity signals received from separate detectors placed at different locations in relation to the subject, and signals corresponding to measured intensity of different wavelengths of light.

At step 1004, monitoring system 310 may generate a cross-correlation signal based on the physiological signals. In some embodiments, monitoring system 310 may compare any two of the physiological signals to generate a cross-correlation signal. In some instances, monitoring system 310 may compare two physiological signals received from detectors placed at different locations and generate a cross-correlation signal based on the comparison using any suitable cross-correlation techniques. In some instances, monitoring system 310 may compare two physiological signals received from the same detector and generate a cross-correlation signal based on the comparison. In some instances, monitoring system 310 may average signals received at the same detector, compare the average signal at one detector to the average signal at another detector, and generate a cross-correlation signal based on the comparison. In any of these instances, the resulting cross-correlation signal may exhibit the common respiratory modulation between the physiological signals.

At step 1006, monitoring system 310 may extract a pulsatile component from the cross-correlation signal. Once the cross-correlation signal is generated, monitoring system 310 may extract a pulsatile component from the cross-correlation signal in accordance with the embodiments described above with respect to step 804.

At step 1008, monitoring system 310 may determine respiration information based on the physiological signals and on the pulsatile component. Once it extracts the pulsatile component in step 1006, monitoring system 310 may determine respiration information in accordance with any of the embodiments described above with respect to step 806. For example, monitoring system 310 may generate respiration morphology signals, autocorrelation sequences, and determine respiration thereon in accordance with any of the embodiments described with respect to step 806 above.

As described above with respect to method 800, monitoring system 310 may perform the additional step of determining a value indicative of oxygen saturation in a region of the subject's tissue (e.g., rSO₂) based on the physiological signals.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A regional oximetry system comprising:
an input for receiving a plurality of physiological signals responsive to regional oxygen saturation in a region of a subject's tissue; and
a processor configured to perform operations comprising:
determining whether the plurality of physiological signals contain a pulsatile component representing the subject's physiological pulse, and
when it is determined that the pulsatile component is present, determining respiration information based at least in part on the pulsatile component.

2. The system of claim 1, wherein the respiration information comprises respiration rate.

3. The system of claim 2, wherein the processor is further configured to perform operations comprising:
determining a period associated with the pulsatile component; and
determining the respiration rate based at least in part on the period.

4. The system of claim 1, wherein the respiration information comprises respiration effort.

5. The system of claim 4, wherein the processor is further configured to perform operations comprising:
determining an amplitude of the pulsatile component; and
determining the respiration effort based at least in part on the amplitude.

6. The system of claim 1, wherein the processor is further configured to perform operations comprising:
determining confidence information associated with each of the plurality of physiological signals;
selecting at least one of the plurality of physiological signals based at least in part on the confidence information; and
determining respiration information based at least in part on the selected physiological signals and on the pulsatile component.

7. The system of claim 1, wherein the processor is further configured to perform operations comprising:
combining at least two of the plurality of physiological signals to generate a combined signal; and
determining respiration information based at least in part on the combined signal and on the pulsatile component.

8. The system of claim 1, wherein the processor is further configured to determine a value indicative of total regional oxygen saturation in a region of the subject's tissue based at least in part on the plurality of physiological signals.

9. The system of claim 1, wherein the processor is further configured to perform operations comprising:
determining whether there is a reliable pulsatile component;
when it is determined that there is a reliable pulsatile component, determining respiration information based at least in part on the reliable pulsatile component.

10. The system of claim 1, wherein the processor is further configured to determine respiration information based at least in part on the plurality of physiological signals.

11. The system of claim 1, wherein the processor is further configured to perform operations comprising:
determining morphology metrics associated with the pulsatile component; and
determining respiration information based at least in part on the morphology metrics.

12. A system comprising:
an input for receiving a plurality of physiological signals generated by a plurality of optical detectors, wherein the plurality of physiological signals are responsive to total oxygen saturation in a region of a subject's tissue; and
a processor configured to perform operations comprising:
extracting a pulsatile component from at least two of the plurality of physiological signals by performing a cross-correlation operation; and
determining respiration information based at least in part on the pulsatile component.

13. The system of claim 12, wherein the respiration information comprises respiration rate.

14. The system of claim 13, wherein the processor is further configured to perform operations comprising:
determining a period associated with the pulsatile component; and
determining the respiration rate based at least in part on the period.

15. The system of claim 12, wherein the respiration information comprises respiration effort.

16. The system of claim 15, wherein determining respiration information further comprises the steps of:
determining an amplitude associated with the pulsatile component; and
determining the respiration effort based at least in part on the amplitude.

17. The system of claim 12, wherein the plurality of physiological signals comprises a first signal indicative of a first depth of penetration and a second signal indicative of a second depth of penetration, and wherein extracting the pulsatile component further comprises the steps of:
comparing the first signal to the second signal;
generating a cross-correlation signal based at least in part on the comparison; and
extracting a pulsatile component from the cross-correlation signal.

18. The system of claim 12, wherein the processor is further configured to determine a value indicative of oxygen saturation in a region of the subject's tissue based at least in part on the plurality of physiological signals.

19. The system of claim 12, wherein the processor is further configured to determine respiration information based at least in part on the plurality of physiological signals.

20. A system comprising:
an input for receiving two pairs of physiological signals, a first pair generated by a first optical detector located at a first location on a subject, and a second pair generated by a second optical detector located at a second location on the subject, the first pair responsive to emitted radiation at two distinct wavelengths and the second pair responsive to emitted radiation at two distinct wavelengths, wherein the first pair of physiological signals and the second pair of physiological signals are also responsive to oxygen saturation in a region of a subject's tissue through which the emitted radiation translates; and
a processor configured to perform operations comprising:
extracting a baseline component from at least one of the physiological signals; and
analyzing the baseline component to determine respiration information.

21. The system of claim 20, wherein the processor is further configured to perform the step of combining at least two of the physiological signals, and wherein extracting a baseline component further comprises extracting a baseline component from the combined physiological signals.

22. The system of claim 20, wherein the processor is further configured to determine a value indicative of oxygen saturation in a region of the subject's tissue based at least in part on the plurality of physiological signals.
